(19) United States
(12) Patent Application Publication
(10) Pub. No.: US 2026/0130954 A1
(43) Pub. Date: May 14, 2026

(54) COMPOUND ADDITIVE FOR FAST-INDUCED PHYSIOLOGICAL REMODELING OF LAYING HENS AND APPLICATION THEREOF

(71) Applicant: Henan Agricultural University, Zhengzhou (CN)

(72) Inventors: Ruirui JIANG, Zhengzhou (CN); Xiangtao KANG, Zhengzhou (CN); Yujie GONG, Zhengzhou (CN); Yadong TIAN, Zhengzhou (CN); Donghua LI, Zhengzhou (CN); Yujie GUO, Zhengzhou (CN); Yanhua ZHANG, Zhengzhou (CN); Yujian LU, Zhengzhou (CN); Shizheng ZHOU, Zhengzhou (CN); Weihua TIAN, Zhengzhou (CN); Zhuanjian LI, Zhengzhou (CN); Wenting LI, Zhengzhou (CN); Hong LI, Zhengzhou (CN); Guirong SUN, Zhengzhou (CN); Ruili HAN, Zhengzhou (CN); Guoxi LI, Zhengzhou (CN)

(73) Assignee: Henan Agricultural University, Zhengzhou (CN)

(21) Appl. No.: 19/364,592

(22) Filed: Oct. 21, 2025

(30) Foreign Application Priority Data
Oct. 24, 2024 (CN) 202411493177.X

(51) Int. Cl. A61K 35/741; A01K 45/00; A61D 19/00; A61K 31/675; A61K 35/00

(52) U.S. Cl. CPC A61K 35/741; A01K 45/00; A61D 19/00; A61K 31/675; A61K 2035/115

(57) ABSTRACT

The present disclosure disclosed a compound additive for FIPR of laying hens and application thereof, and relates to the technical field of FIPR. Using the compound additive provided by the present disclosure in conjunction with fasting, the expression levels of intestinal barrier-related genes and intestinal proliferation-related genes can be significantly increased. The compound additive and method can significantly increase the expression levels of IgA, IgM and IgG, thereby improving the immune performance of laying hens. With the improvement of intestinal health and immunity, the FIPR of laying hens is successfully achieved: the recovery ability of weight and laying rate of laying hens after fasting, and the color depth of egg yolk on the 30^{th} day of resumption after fasting are significantly better than those of the control group. This provides a technical guarantee for prolonging the breeding period of laying hens, saving costs and increasing efficiency.

### TECHNICAL FIELD

The present disclosure relates to the technical field of fast-induced physiological remodeling (FIPR), particularly a compound additive for FIPR of laying hens and application thereof.

### BACKGROUND

In recent years, the extended breeding of laying hens has become a global trend as feed ingredient prices, such as corn and soybean meal, have continued to rise. During the 14th Five-Year Plan period, a key objective for China's laying hens industry technology system is to extend the breeding cycle, to achieve 500 eggs in 700 days, and to ensure high laying rates and quality during the late laying stage. FIPR is one of the primary approaches to achieve this goal.

FIPR is a method to induce the remodeling of various functions of laying hens through fasting, in which feather loss and rejuvenation (commonly known as induced molting) are the prominent features in this process. In the process of FIPR, the physiology and metabolism of laying hens will change significantly. For example, there is a regulatory effect on follicular development. The ovarian function and follicular development of laying hens will be changed through fasting, so as to promote the development of primary and secondary follicles, thereby extending the laying cycle of laying hens and achieving a second laying peak for the hens. The bile acid synthesis, secretion, and uptake are affected. And the fatty liver caused by metabolic disorders in aged laying hens is effectively alleviated. Nevertheless, during the FIPR process, immunosuppression in laying hens is also induced, which increases susceptibility to pathogenic bacteria such as Salmonella enteritidis and Escherichia coli, leading to issues like intestinal flora disorder and severe intestinal injury, while the corresponding intestinal health care protocols remain limited. Further studies have found that the intestinal structure is disrupted in the process of inducing physiological remodeling, and the content of intestinal beneficial bacteria Bacteroidetes and its anabolic product vitamin B6 decreases significantly.

### SUMMARY

An objective of the present disclosure is to provide a compound additive for FIPR of laying hens and application thereof, to solve the problems of immunosuppression, intestinal flora disorder, and severe intestinal injury, which are easily induced during the process of FIPR of laying hens in the prior art.

In order to achieve the above objective, the present disclosure provides a compound additive for FIPR of laying hens, the compound additive includes Bacteroides fragilis ATCC25285+vitamin B6, a number of viable bacteria of Bacteroides fragilis ATCC25285 in the compound additive is 1×10⁹ CFU/mL, and a mass of vitamin B6 is 0.5 mg.

In some embodiments, Bacteroides fragilis ATCC25285 is activated with a brain-heart infusion broth medium, and the pH of the medium is 7.4±0.2.

A method for FIPR of laying hens, applying the aforementioned compound additive for FIPR of laying hens, the method includes the following steps:
S1, performing a fasting induction by selecting healthy laying hens in a late laying stage, stopping the water and feed supply on 1st to 3rd days of fasting, exposing the laying hens to light for 8 h every day, and avoiding a flow of staff and noise pollution beside a chicken coop;
S2, on 4th to 15th days of fasting, continuously stopping feed supply, supplying water normally, administering the compound additive for FIPR of laying hens, and exposing the laying hens to light for 8 h every day;
S3, when a weight loss rate of laying hens is >25%, resuming the feeding for the laying hens, and gradually prolonging a light exposure time for 30 min every day until 16 h, and stopping the prolonging after 16 h.

In some embodiments, in S2, 1 mL of the the compound additive for FIPR of laying hens is administered to each laying hen.

In some embodiments, in S2, the fasting is terminated early when a mortality rate of the laying hens is equal to or greater than 4%.

In some embodiments, surroundings of the chicken coop are sterilized daily during fasting.

In some embodiments, in S3, the method for resuming the feeding includes: feeding 30 g of basal diet for each laying hen daily on 1^{st} to 2^{nd} days after resuming the feeding; feeding 60 g of basal diet for each laying hen daily on 3^{rd} to 4^{th} days after resuming the feeding; feeding 90 g of basal diet for each laying hen daily on a 5th day after resuming the feeding; and feeding 120 g of basal diet for each laying hen daily on 6^{th} to 20^{th} days after resuming the feeding; after this stage, the laying hens are allowed free feeding.

An application for a compound additive for the FIPR of laying hens as described above in the FIPR of laying hens.

Bacteroides fragilis ATCC25285 is considered to be a beneficial bacterium and an important part of the intestinal flora, which is conducive to maintaining intestinal homeostasis. It plays a crucial role in promoting the maturation of the immune system, inhibiting intestinal inflammation, and improving the structure of intestinal flora. It is commonly found in the human intestinal flora and also exists in the oral cavity and upper respiratory tract. Vitamin B6 is an essential micronutrient to maintain the healthy growth of animals, exhibiting physiological functions such as anti-inflammatory, anti-oxidation, neuromodulation and anti-tumor. Among the reports on Bacteroides fragilis ATCC25285 and vitamin B6, there are no studies on the combined application of Bacteroides fragilis ATCC25285 and vitamin B6.

Therefore, the present disclosure provides a compound additive for FIPR of laying hens and application thereof, and the specific technical effects thereof are as follows:

(1) By adopting the compound additive and the method provided by the present disclosure, the FIPR of laying hens can be successfully achieved: the weight and laying rate recovery ability of laying hens after fasting, and the color depth of egg yolk on the 30^{th} day of resuming the feeding after fasting are significantly better than those of the control group, thus providing technical support for prolonging the breeding period of laying hens, saving costs and increasing efficiency.

(2) By adopting the compound additive and the method provided by the present disclosure, the ileum villus height, the expression level of intestinal barrier-related genes and intestinal proliferation-related genes of laying hens can be significantly improved, the expression level of intestinal pro-inflammatory-related genes can be significantly reduced, and the intestinal stress injury and inflammation can be alleviated, thereby improving the intestinal health degree of laying hens significantly.

(3) By adopting the compound additive and the method provided by the present disclosure, the expression levels of immunoglobulin A (IgA), immunoglobulin M (IgM) and immunoglobulin G (IgG) can be significantly improved, thereby improving the immune performance of laying hens.

### BRIEF DESCRIPTION OF THE DRAWINGS

To explain the embodiments of the present disclosure or the technical solutions in the prior art more clearly, a brief introduction will be made to the accompanying drawings used in the embodiments or the description of the prior art. It is obvious that the drawings in the description below are only some embodiments of the present disclosure, and those ordinarily skilled in the art can obtain other drawings according to these drawings without creative work.

FIG. 1 shows statistical results of laying rates at different fasting stages in an embodiment of the present disclosure;

FIG. 2 is a slice diagram (H.E. 5x) of the morphological tissue of jejunum of laying hens at different stages according to an embodiment of the present disclosure;

FIG. 3 is a slice diagram (H.E. 5x) of the morphological tissue of ileum of laying hens at different stages according to an embodiment of the present disclosure;

FIG. 4 shows an expression level of jejunum-related genes in laying hens according to an embodiment of the present disclosure, wherein part A is a relative expression level of TNF-α at different stages, part B is a relative expression level of IL-15 at different stages, part C is a relative expression level of IL-6 at different stages, part D is a relative expression level of IL-18 at different stages, part E is a relative expression level of Claudin-1 at different stages, part F is a relative expression level of occludin-1 at different stages, part G is a relative expression level of Lgr5 at different stages, and part H is the relative expression level of CDK1;

FIG. 5 shows an expression level of ileum-related genes in laying hens according to an embodiment of the present disclosure, wherein part A is a relative expression level of TNF-α at different stages, part B is a relative expression level of IL-15 at different stages, part C is a relative expression level of IL-6 at different stages, part D is a relative expression level of IL-18 at different stages, part E is a relative expression level of Claudin-1 at different stages, part F is a relative expression level of occludin-1 at different stages, part G is a relative expression level of Lgr5 at different stages, and part H is the relative expression level of CDK1.

### DETAILED DESCRIPTION OF THE EMBODIMENTS

The technical scheme of the present disclosure is further explained below by drawings and embodiments.

In order to make the objectives, technical solutions, and advantages of the present application clearer, more thorough, and more complete, the following description provides a clear and complete explanation of the technical solutions of the present disclosure with reference to the accompanying drawings and embodiments. The following detailed description is an illustration of the embodiments, intended to provide a further detailed explanation of the present disclosure. Unless otherwise specified, all technical terms used herein have the same meanings as those commonly understood by those skilled in the art to which the present application pertains.

The instruments, equipment, reagents, and materials used in the examples are commercially available. Unless otherwise specified, the experimental methods described in in the embodiments are conventional methods within the field.

### Embodiment 1

The FIPR for laying hens is performed, with the specific settings detailed in Table 1. The steps are as follows:

TABLE 1

| | Processing | | Drinking | | Sampling |
|---|---|---|---|---|---|
| Stage | stage/d | Feeding | water | Light | time point |
| Environmental | 1-10 | 120 g/d | Free | 16 h | 1 day before |
| adaptation stage | | | drinking | | fasting (F0) |
| | | | water | | |
| Fasting | 1-3 | Stopping | Stopping | 8 h | Fasting for 3 |
| induction stage | | feed | water | | days (F3) |
| | | supply | supply | | |
| | 4-15 | Stopping | Free | 8 h | Fasting for 15 |
| | | feed | drinking | | days (F15) |
| | | supply | water | | |
| Resuming | 1-2 | 30 g/d | Free | 8 h | Resuming |
| feeding stage | | | drinking | | feeding for 5 |
| | | | water | | days (R5) |
| | 3-4 | 60 g/d | Free | Increase | |
| | | | drinking | by 0.5 h | |
| | | | water | daily | |
| | 5 | 90 g/d | Free | until | |
| | | | drinking | reaching | |
| | | | water | 16 h | |
| | 6-20 | 120 g/d | Free | | Resuming |
| | | | drinking | | feeding for 15 |
| | | | water | | days (R15) |
| | | | Free | | |
| | | | drinking | | |
| | | | water | | |
| 2^{nd} laying | 21-30 | 120 g/d | Free | 16 h | Resuming |
| stage | | | drinking | | feeding for 30 |
| | | | water | | days (R30) |

S11, 288 healthy, disease-free 420-day-old short-footed houdan chicken with weights of 1300-1500 g are selected and randomly divided into 4 groups, each with 9 replicates, with 8 chickens in each replicate and 72 chickens in each group. One group is the control, while the other three are treatment groups: the Bacteroides fragilis ATCC25285 group, the Vitamin B6 group, and the Bacteroides fragilis ATCC25285+Vitamin B6 compound additive group.

S12, the experimental chicken coop is strictly sterilized using formaldehyde and potassium permanganate fumigation. Then, the laying hens selected in step S11 are transferred to the chicken coop and routinely fed the basal diet (acclimation period) for 10 days. The composition and nutritional levels of the basal diet are shown in Table 2.

TABLE 2

| Project | % | Nutrition level | % |
|---|---|---|---|
| Corn | 62.50 | Metabolic energy | 11.20 |
| Soybean meal | 25.50 | Crude protein | 15.80 |
| Stone powder | 9.20 | Calcium | 3.78 |
| Oil powder | 0.80 | Available phosphorus | 0.28 |
| Premix | 2.00 | Lysine | 0.71 |
| Total | 100.00 | Methionine | 0.66 |

Wherein the premix is provided per kilogram of feed as follows: 12,000 IU of Vitamin A, 3,000 IU of Vitamin D3, 30 IU of Vitamin E, 10 mg of Vitamin K, 12 mg of Vitamin B1, 0.05 mg of Vitamin B12, 16.4 mg of biotin, 30 mg of niacin, 10 mg of pantothenic acid, 1 mg of folic acid, 500 mg of choline, 45 mg of Cu, 80 mg of Fe, 85 mg of Zn, 70 mg of Mn, 0.5 mg of I, and 0.0 mg of Se. Crude protein and calcium are the measured values; the remaining nutrient levels are calculated values.

S13, then 1% seashell powder is added to the basic diet, and multivitamins (Yipan) are added to the drinking water for 10 days. The addition of 1% seashell powder can reduce egg breakage rates after the start of molting and prevent calcium depletion in chickens post-molting. The addition of multivitamins in the drinking water can prevent stress reactions.

S14, the chickens in each treatment group are numbered. 10 chickens are randomly selected from each treatment group for subsequent monitoring of weight changes to estimate the overall weight changes of the flock. Before the day of fasting induction (F0), the weight and laying numbers of the control and experimental groups of laying hens are weighed and recorded, respectively. The laying rate (total number of laying eggs/total number of laying hens) is calculated. The feeders are cleaned, and the water is drained from the water supply lines.

The weight of the laying hens in the F0 generation is the average weight of 40 randomly selected laying hens from the four treatment groups. The laying rate of the F0 generation is the average laying rate of the 288 laying hens in the experiment. The average weight of the F0 generation is 1248 g, and the laying rate is 37.35%.

S15, fasting induction stage (F) settings are shown in Table 1:

(1) During the 1^{st} to 3^{rd} days of the fasting induction stage, water and feed (basal diet) are stopped. 8 h of light exposure is maintained. During this stage, staff in the chicken coop should minimize movement, maintain a quiet environment, and avoid any external factors beyond the stopping of water and feed.

(2) From the 4^{th} to 15^{th} days of fasting induction, feed is stopped, but water is supplied. The control group is administered a saline solution. The Bacteroides fragilis ATCC25285 group is administered a Bacteroides fragilis ATCC25285 viable bacteria additive with the number of viable bacteria of 1×10⁹ CFU daily. The vitamin B6 group is administered 0.5 mg of vitamin B6 daily. The compound additive group is administered Bacteroides fragilis ATCC25285 viable bacteria additive with the number of viable bacteria of 1×10⁹ CFU+0.5 mg of vitamin B6 daily, with 8 h of light exposure.

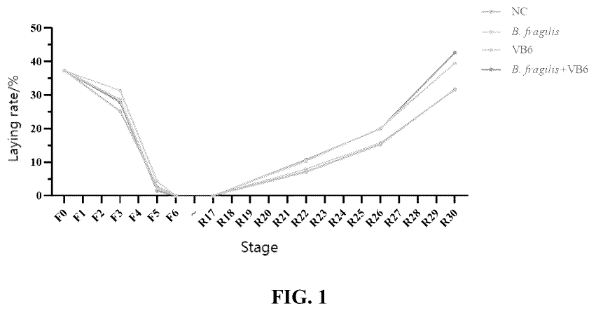

S16, the weights of laying hens in the control and treatment groups are weighed and recorded at 4:00 PM every three days after the start of the fasting induction. Weight loss rates are calculated using the following formula (results shown in Table 4), and laying rates are counted and recorded (results shown in FIG. 1). When the average weight loss rate of the laying hen group reaches 25%, feeding is resumed. Weight loss rates are calculated using the following formula:

Weight loss rate at Fx (%):(Weight at F0-Weight at Fx)/Weight at F0×100%, where F denotes the fasting period, F0 is the day before fasting starts, and Fx is the x-th day of fasting.

Based on monitoring of the weight of the flock, the fasting period is determined to be 15 days.

S17, resumption of feeding stage (R). On the 16^{th} day of the fasting induction stage (recorded as the 1^{st} day of the resumption of feeding stage, R1), the hens are entered into the resumption of feeding stage. During this stage, the flock maintains normal water supply. Feeding conditions during the resumption of the feeding stage are as follows:
30 g of basal diet for each laying hen is fed daily on the 1^{st} to 2^{nd} days after resuming the feeding;
60 g of basal diet for each laying hen is fed daily on the 3^{rd} to 4^{th} days after resuming the feeding;
90 g of basal diet for each laying hen is fed daily on the 5^{th} day after resuming the feeding;
120 g of basal diet for each laying hen is fed daily on the 6^{th} to 20^{th} days after resuming the feeding;
after this stage, the laying hens are allowed free feeding.

The light exposure is increased by 30 min daily during the gradual resumption of feeding until reaching 16 h/day.

### Embodiment 2

The production performance and egg quality of laying hens shall be evaluated on the day before fasting, the fasting induction stage, and the resumption of feeding stage. The specific steps are as follows:

S21, the number of laying of laying hens in each group is recorded daily at 4:00 PM. The average daily laying rate for each of the five stages of F0-F3, F4-F5, R17-R22, R23-R26, and R27-R30 is then taken as the laying rate for those respective stages. The weight of each group is recorded every three days, and the weight loss rate is calculated.

Weight loss rate at Rx (%)=(weight at F0-weight at Rx)/weight at F0×100%, where R denotes the resumption of feeding stage, F0 denotes the day before the start of fasting, and Rx denotes the x-th day of resumption of feeding.

On the R30 stage, specifically the 30^{th} day after the end of the fasting stage, 25 eggs are randomly collected from each group for egg quality parameter measurements. A multifunctional egg quality analyzer is used for the measurement, and the measured indicators include egg weight, egg shape indicator, eggshell thickness, eggshell strength, eggshell weight, albumen height, and yolk color.

### Embodiment 3

The immune function, intestinal tissue morphology, and expression levels of intestinal-related genes of laying hens are evaluated on the day before fasting, the fasting induction stage, and the refeeding recovery stage. The specific steps are as follows:

S31, at F0, F15, R5, and R30, one laying hen with an approximate average weight is selected from each replicate in both the treatment and control groups, 9 replicates in total. Blood is collected from the wing vein using a coagulation tube. Serum is collected after centrifugation at 1265×g for 10 min. The serum is frozen rapidly in liquid nitrogen and transferred to −80° C. for storage, which is used for serum immune indicators detection.

The contents of IgA, IgM and IgG are detected by ELASA kit.

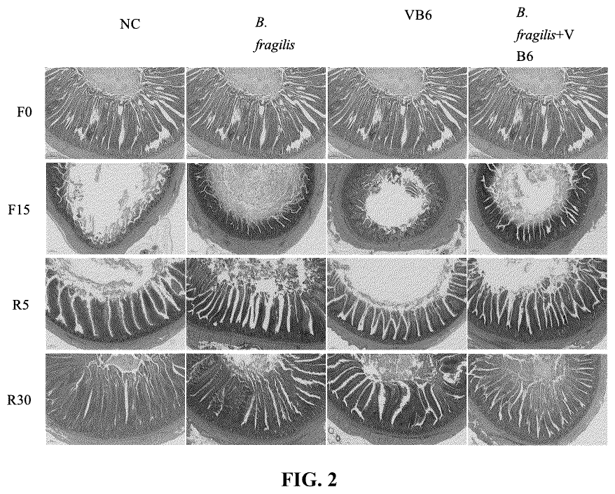

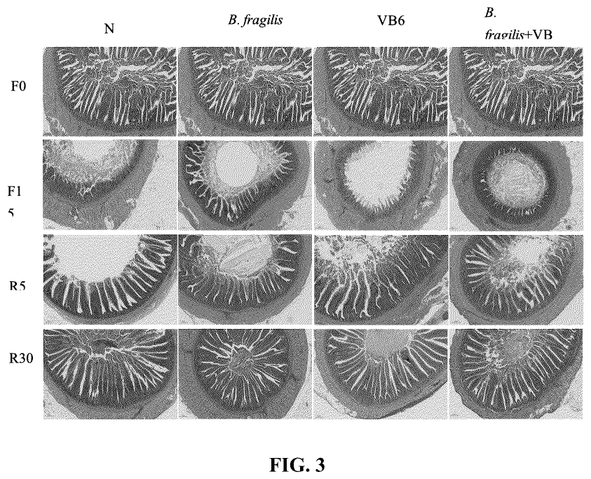

S32, after blood collection, the experimental chickens are slaughtered. The small intestine of 1 cm jejunum and ileum is collected and washed slowly with 0.9% saline solution, and then stored in a 10 mL centrifuge tube containing 4% paraformaldehyde (fixative) for fixation. After 24 h of fixation of the jejunum and ileum segments to be collected, the intestinal segments are taken out from the fixative and embedded in paraffin, made into slices, and stained with hematoxylin-eosin (HE), observed, and photographed under an optical microscope. The results are shown in FIGS. 2 and 3. The villus height and crypt depth of each intestinal segment are measured by Motic DSAssistant Lite software, and the villus/crypt ratio (villus height/crypt depth) is calculated. The results are shown in Table 6.

S33, the jejunum and ileum tissue samples are collected and placed in a 1.5 mL sterile non-enzymatic centrifuge tube, frozen quickly in liquid nitrogen, and finally stored in a refrigerator at −80° C. for subsequent measurement.

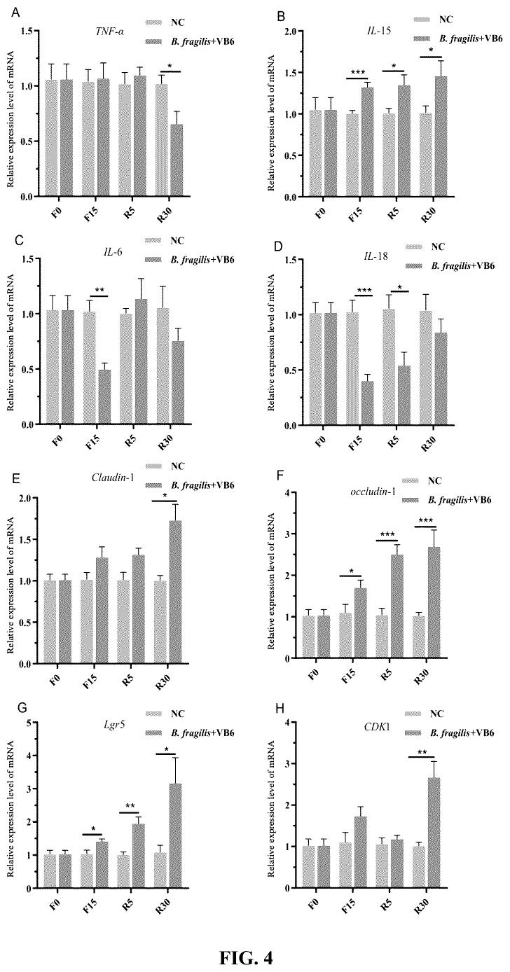

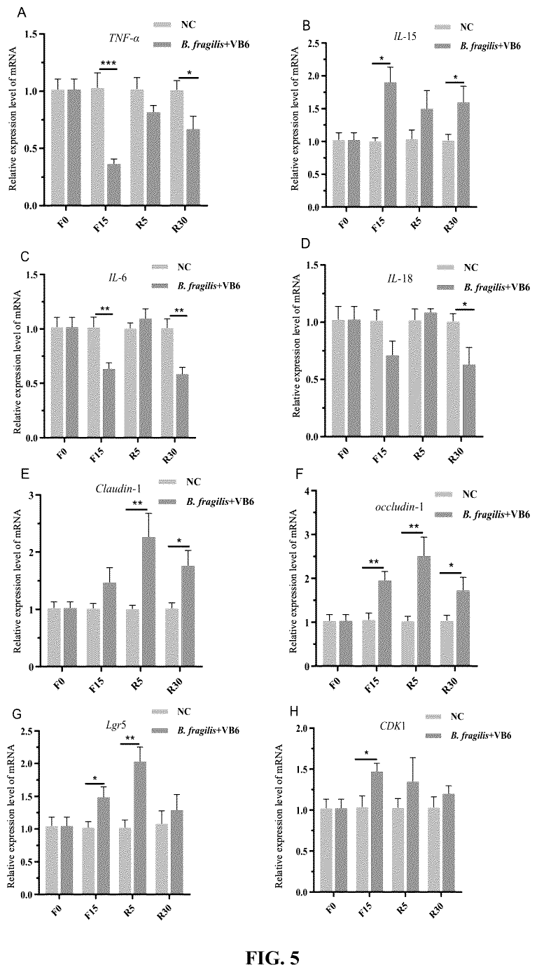

The total RNA of jejunum and ileum of each sampling point is extracted by kit. The glyceraldehyde-3-phosphate dehydrogenase (GAPDH) is used as the internal reference gene. The target gene is detected by quantitative Real-time polymerase chain reaction (PCR), and the 2^{-ΔΔCt} method is used to calculate the relative expression level of mRNA of the target gene in the intestinal segment. The results are shown in FIGS. 4 and 5. The primer sequence and fragment size information of the internal reference gene and the target gene are shown in Table 3.

TABLE 3

| | Accession | | Product |
|---|---|---|---|
| Gene | number | Primer sequence (5'-3') | size/bp |
| Tumor necrosis | NM_204267.2 | F: GCCCAGTTCAGATGAGTTGCC | 169 |
| factor-α/TNF-α | | R: AAGAGGCCACCACACGACAG | |
| Interleukin-15/ | NM_204571.2 | F: CTCCATAGGTTTCCGAGGCTTGT | 159 |
| IL-15 | | R: ATGTGTTTTCTGACTCTCCGGC | |
| Interleukin-6/ | NM_204628.2 | F: AAATGTCCAGCAGCCAGACT | 130 |
| IL-6 | | R: CTTTCGGAGCGGCCTTCATA | |
| Interleukin-18/ | XM_046932263.1 | F: TGATGAGCTGGAATGCGATG | 148 |
| IL-8 | | R: ACTGCCAGATTTCACCTCCTG | |
| occlidin-1 | XM_046904540.1 | F: CCGTGGAGTCCAGTGATGAG | 123 |
| | | R: TCGAACTCCTGCTTGTAGCG | |
| Claudin-1 | NM_001013611.2 | F: CACCCGTTAACACCAGATTTGA | 100 |
| | | R: AGGGGGCATTTTTGGGGTAG | |
| Leucine-rich | XM_046909876.1 | F: ACGTCTTGCAGGAAATGGCT | 159 |
| repeat-containing | | R: TTGGCATCCAGGCGTAGAGA | |
| G-protein coupled | | | |
| receptor 5/Lgr5 | | | |
| Cyclin-dependent | NM_205314.2 | F: TAATAGATGACAAAGGGGT | 130 |
| kinase 1/CDK1 | | R: GAGTGGAATACAGAGCAGA | |
| Glyceraldehyde- | NM_205518 | F: TGTTACCAACACCCACACCC | 110 |
| 3-phosphate | | R: TCCTGAGTCAAGCGCCAAAA | |
| dehydrogenase/ | | | |
| GAPDH | | | |

### Results Analysis

The experimental data are analyzed by Excel 2010, and the data are analyzed by SPSS 26.0 software. The production performance and intestinal tissue morphology are compared by single-factor analysis of variance and Duncan's method. The intestinal gene expression and serum immune indicators are analyzed by the t-test. The results are expressed as ‘mean±standard deviation’, and P<0.05 indicates a significant difference. GraphPad Prism 8.0 software is used for data and graphics processing.

The shoulder label of peer data in the table shows significant differences in different lowercase letters (P<0.05). The control group is represented by NC, the B. fragilis ATCC25285 group is represented by B. fragilis, vitamin B6 group is represented by VB6, B. fragilis ATCC25285 and vitamin B6 compound additive group is represented by B. fragilis+VB6.

(1) The statistical results of the weight loss rate (%) of laying hens in the experimental group and the control group are shown in Table 4.

The weight recovery of the Bacteroides fragilis ATCC25285+vitamin B6 compound additive group is better than that of other groups on the 45^{th} day (R30), but there is no significant difference between the groups at each stage (P>0.05).

TABLE 4

| | Groups | | | | |
|---|---|---|---|---|---|
| | NC | B. Fragilis | VB6 | B. Fragilis + | P |
| Time | (%) | (%) | (%) | VB6 (%) | values |
| Day 3 | 0.12 ± 0.04 | 0.15 ± 0.02 | 0.11 ± 0.03 | 0.14 ± 0.06 | 0.237 |
| Day 6 | 0.16 ± 0.05 | 0.18 ± 0.02 | 0.16 ± 0.03 | 0.19 ± 0.05 | 0.585 |
| Day 9 | 0.21 ± 0.06 | 0.21 ± 0.02 | 0.2 ± 0.03 | 0.22 ± 0.05 | 0.771 |
| Day 12 | 0.24 ± 0.06 | 0.24 ± 0.02 | 0.24 ± 0.02 | 0.26 ± 0.04 | 0.913 |
| Day 15 | 0.27 ± 0.07 | 0.28 ± 0.02 | 0.28 ± 0.03 | 0.28 ± 0.04 | 0.947 |
| Day 18 | 0.27 ± 0.06 | 0.24 ± 0.01 | 0.30 ± 0.04 | 0.29 ± 0.05 | 0.209 |
| Day 21 | 0.24 ± 0.05 | 0.22 ± 0.04 | 0.26 ± 0.05 | 0.24 ± 0.04 | 0.487 |
| Day 24 | 0.21 ± 0.05 | 0.18 ± 0.02 | 0.23 ± 0.06 | 0.19 ± 0.04 | 0.338 |
| Day 27 | 0.14 ± 0.04 | 0.12 ± 0.03 | 0.15 ± 0.04 | 0.13 ± 0.04 | 0.442 |
| Day 30 | 0.11 ± 0.03 | 0.10 ± 0.04 | 0.13 ± 0.04 | 0.13 ± 0.05 | 0.663 |
| Day 33 | 0.10 ± 0.02 | 0.08 ± 0.05 | 0.13 ± 0.06 | 0.10 ± 0.05 | 0.495 |
| Day 36 | 0.07 ± 0.01 | 0.06 ± 0.05 | 0.10 ± 0.09 | 0.07 ± 0.06 | 0.819 |
| Day 39 | 0.07 ± 0.03 | 0.05 ± 0.06 | 0.09 ± 0.10 | 0.03 ± 0.07 | 0.488 |
| Day 42 | 0.07 ± 0.04 | 0.03 ± 0.06 | 0.08 ± 0.10 | 0.01 ± 0.06 | 0.309 |
| Day 45 | 0.03 ± 0.03 | 0.02 ± 0.07 | 0.09 ± 0.07 | 0.00 ± 0.06 | 0.127 |

(2) The effects of Bacteroides fragilis ATCC25285 and vitamin B6 on the laying rate of laying hens during FIPR are shown in FIG. 1. During F1-F3, the average laying rate of NC group decreased to 25.51%, the average laying rate of Bacteroides fragilis ATCC25285 group decreased to 28.70%, the average laying rate of vitamin B6 group decreased to 31.48%, and the average laying rate of Bacteroides fragilis ATCC25285+vitamin B6 group decreased to 27.89%. During F4-F5, the average laying rate of the NC group decreased to 2.55%, the average laying rate of the Bacteroides fragilis ATCC25285 group decreased to 2.09%, the average laying rate of the vitamin B6 group decreased to 4.23%, and the average laying rate of the Bacteroides fragilis ATCC25285+vitamin B6 group decreased to 1.14%. During F6-R17, chickens began to enter the cease period. The NC group, Bacteroides fragilis ATCC25285 group, vitamin B6 group, and Bacteroides fragilis ATCC25285+vitamin B6 group began to lay eggs again at R20, R18, R21, and R19, respectively, and the laying rate of each group gradually increased. In R17-R22, the average laying rate of the NC group is 7.08%, the average laying rate of the Bacteroides fragilis ATCC25285 group is 10.37%, the average laying rate of the vitamin B6 group is 7.96%, and the average laying rate of the Bacteroides fragilis ATCC25285+vitamin B6 compound additive group is 10.71%. In R23-R26, the average laying rate of NC group is 15.27%, the average laying rate of Bacteroides fragilis ATCC25285 group is 20.21%, the average laying rate of vitamin B6 group is 15.91%, and the average laying rate of Bacteroides fragilis ATCC25285+vitamin B6 compound additive group is 20.07%. In R27-R30, the average laying rate of NC group is 31.83%, the average laying rate of Bacteroides fragilis ATCC25285 group is 39.54%, the average laying rate of vitamin B6 group is 31.17%, and the average laying rate of Bacteroides fragilis ATCC25285+vitamin B6 compound additive group is 42.64%.

(3) The effects of Bacteroides fragilis ATCC25285 and vitamin B6 on egg quality during FIPR are shown in Table 5. Compared with R30NC, the yolk color of Bacteroides fragilis ATCC25285+vitamin B6 compound additive group is significantly deeper (P<0.05). The eggshell weight and eggshell strength of the vitamin B6 group are significantly increased (P<0.05), and there is no significant difference in egg weight, egg shape indicator and albumen height between the groups (P>0.05).

TABLE 5

| | Groups | | | | |
|---|---|---|---|---|---|
| | R30 | R30 | R30 | R30 | P |
| Items | NC | B. fragilis | VB6 | B. fragilis + VB6 | values |
| Egg weight/g | 51.54 ± 5.11 | 50.51 ± 3.86 | 51.80 ± 5.08 | 50.58 ± 5.25 | 0.094 |
| Egg shape | 1.34 ± 0.07^{ab} | 1.37 ± 0.09^{a} | 1.32 ± 0.06^{b} | 1.35 ± 0.06^{ab} | 0.081 |
| indicator | | | | | |
| Eggshell | 0.32 ± 0.04 | 0.32 ± 0.04 | 0.31 ± 0.03 | 0.31 ± 0.04 | 0.455 |
| thickness/mm | | | | | |
| Eggshell | 35.42 ± 8.16^{b} | 36.26 ± 8.81^{b} | 40.63 ± 7.7^{a} | 35.78 ± 7.56^{b} | 0.090 |
| strength/N | | | | | |
| Eggshell | 6.82 ± 0.83^{b} | 6.61 ± 0.42^{b} | 7.45 ± 0.9^{a} | 6.88 ± 0.97^{b} | 0.004 |
| weight/g | | | | | |
| Albumen | 4.64 ± 0.92 | 4.60 ± 0.73 | 4.31 ± 1.04 | 4.66 ± 0.81 | 0.481 |
| height/mm | | | | | |
| Yolk color | 8.80 ± 0.83^{b} | 9.31 ± 0.55^{a} | 9.2 ± 0.67^{ab} | 9.41 ± 0.87^{a} | 0.014 |

(4) The effects of Bacteroides fragilis ATCC25285 and vitamin B6 on the intestinal morphology of laying hens during FIPR are shown in Table 6. Compared with the control group, in the F15, the crypt depth of the jejunum in the vitamin B6 group is significantly thickened (P<0.05); the villus height of the ileum in the Bacteroides fragilis ATCC25285+vitamin B6 compound additive group is significantly increased (P<0.05). The villus height, crypt depth and villus/crypt ratio of the intestine in other stages are not significant (P>0.05).

TABLE 6

| | | Groups | | | | |
|---|---|---|---|---|---|---|
| | | | B. | | B. | P |
| Stage | Item | NC | fragilis | VB6 | fragilis + VB6 | values |
| | Jejunum | | | | | |
| F0 | Villus | 850.59 ± 335.70 | 850.59 ± 335.70 | 850.59 ± 335.70 | 850.59 ± 335.70 | 1.000 |
| | height | | | | | |
| | Crypt | 173.96 ± 45.77 | 173.96 ± 45.77 | 173.96 ± 45.77 | 173.96 ± 45.77 | 1.000 |
| | depth | | | | | |
| | Villus/crypt | 5.25 ± 2.50 | 5.25 ± 2.50 | 5.25 ± 2.50 | 5.25 ± 2.50 | 1.000 |
| | ratio | | | | | |
| F15 | Villus | 415.76 ± 131.98 | 549.87 ± 225.53 | 415.73 ± 133.14 | 507.23 ± 104.84 | 0.275 |
| | height | | | | | |
| | Crypt | 187.27 ± 35.07^{b} | 192.57 ± 21.37^{ab} | 240.59 ± 27.34^{a} | 203.14 ± 48.49^{ab} | 0.039 |
| | depth | | | | | |
| | Villus/crypt | 2.34 ± 0.95 | 3.00 ± 1.32 | 1.79 ± 0.68 | 2.68 ± 0.76 | 0.138 |
| | ratio | | | | | |
| R5 | Villus | 860.25 ± 193.67 | 871.36 ± 276.89 | 716.73 ± 174.09 | 814.07 ± 223.83 | 0.498 |
| | height | | | | | |
| | Crypt | 156.96 ± 51.15 | 159.75 ± 55.42 | 137.16 ± 43.29 | 174.42 ± 42.57 | 0.528 |
| | depth | | | | | |
| | Villus/crypt | 6.34 ± 2.76 | 5.89 ± 1.39 | 6.04 ± 1.67 | 4.98 ± 1.08 | 0.552 |
| | ratio | | | | | |
| R30 | Villus | 987.10 ± 226.78 | 1036.96 ± 269.72 | 1256.65 ± 226.04 | 1110.66 ± 478.49 | 0.369 |
| | height | | | | | |
| | Crypt | 179.62 ± 96.46 | 159.06 ± 30.34 | 176.75 ± 41.72 | 165.26 ± 60.52 | 0.901 |
| | depth | | | | | |
| | Villus/crypt | 7.39 ± 3.56 | 7.15 ± 3.09 | 7.91 ± 2.79 | 7.32 ± 3.01 | 0.967 |
| | ratio | | | | | |
| | Ileum | | | | | |
| F0 | Villus | 969.77 ± 138.01 | 969.77 ± 138.01 | 969.77 ± 138.01 | 969.77 ± 138.01 | 1.000 |
| | height | | | | | |
| | Crypt | 143.01 ± 49.57 | 143.01 ± 49.57 | 143.01 ± 49.57 | 143.01 ± 49.57 | 1.000 |
| | depth | | | | | |
| | Villus/crypt | 7.97 ± 3.55 | 7.97 ± 3.55 | 7.97 ± 3.55 | 7.97 ± 3.55 | 1.000 |
| | ratio | | | | | |
| F15 | Villus | 376.98 ± 96.1^{b} | 426.6 ± 90.64^{ab} | 297.71 ± 91.9^{b} | 519.55 ± 144.16^{a} | 0.007 |
| | height | | | | | |
| | Crypt | 166.28 ± 25.9 | 183.04 ± 23.35 | 161.05 ± 40.84 | 183.71 ± 32.76 | 0.392 |
| | depth | | | | | |
| | Villus/crypt | 2.23 ± 0.85 | 2.43 ± 0.45 | 2.23 ± 1.16 | 2.75 ± 1.23 | 0.694 |
| | ratio | | | | | |
| R5 | Villus | 669.09 ± 158.13 | 689.74 ± 202.24 | 589.2 ± 197.22 | 618.23 ± 171.58 | 0.682 |
| | height | | | | | |
| | Crypt | 159.42 ± 53.20 | 144.81 ± 46.04 | 132.05 ± 47.20 | 178.22 ± 45.83 | 0.276 |
| | depth | | | | | |
| | Villus/crypt | 5.09 ± 2.61 | 5.05 ± 0.95 | 4.73 ± 1.18 | 3.88 ± 1.71 | 0.483 |
| | ratio | | | | | |
| R30 | Villus | 797.80 ± 367.83 | 902.22 ± 241.35 | 808.56 ± 328.57 | 937.23 ± 317.72 | 0.789 |
| | height | | | | | |
| | Crypt | 165.72 ± 76.27 | 124.86 ± 38.8 | 150.12 ± 82.71 | 175.13 ± 49.04 | 0.453 |
| | depth | | | | | |
| | Villus/crypt | 5.65 ± 2.27 | 8.13 ± 3.01 | 6.31 ± 2.56 | 5.63 ± 1.60 | 0.168 |
| | ratio | | | | | |

Note:
where the unit of villus height is μm, and the unit of crypt depth is μm.

(5) The effects of Bacteroides fragilis ATCC25285 and vitamin B6 on the immune indicators of laying hens during FIPR are evaluated. The results are shown in Table 7. In the R5 stage, IgA of the Bacteroides fragilis ATCC25285+vitamin B6 compound additive group is significantly higher than that in the control group (P<0.05). In the F15, R5, and R30 stages, IgG of the Bacteroides fragilis ATCC25285+vitamin B6 compound additive group is significantly higher than that in the control group (P<0.05). In the R5 stage, IgM of the Bacteroides fragilis ATCC25285+vitamin B6 compound additive group is significantly higher than that in the control group at R5 (P<0.05).

TABLE 7

| Stage | Groups | IgA/(μg/mL) | IgG/(ng/mL) | IgM/(μg/mL) |
|---|---|---|---|---|
| F0 | NC | 9.29 ± 2.99 | 98.88 ± 2.60 | 6.18 ± 0.23 |
| | B. fragilis + | 9.29 ± 2.99 | 98.88 ± 2.60 | 6.18 ± 0.23 |
| | VB6 | | | |
| | P value | 1.000 | 1.000 | 1.000 |
| F15 | NC | 9.06 ± 0.24 | 84.83 ± 2.26^{b} | 5.30 ± 0.33 |
| | B. fragilis + | 8.83 ± 0.14 | 88.94 ± 2.69^{a} | 5.31 ± 0.32 |
| | VB6 | | | |
| | P value | 0.111 | 0.007 | 0.947 |
| R5 | NC | 9.61 ± 0.27^{b} | 89.75 ± 4.19^{b} | 5.34 ± 0.26^{b} |
| | B. fragilis + | 10.11 ± 0.24^{a} | 99.24 ± 4.97^{a} | 6.00 ± 0.22^{a} |
| | VB6 | | | |
| | P value | 0.015 | 0.001 | <0.001 |
| R30 | NC | 8.93 ± 0.08 | 91.50 ± 2.44^{b} | 5.31 ± 0.28 |
| | B. fragilis + | 8.80 ± 0.31 | 96.53 ± 3.13^{a} | 5.34 ± 0.22 |
| | VB6 | | | |
| | P value | 0.387 | 0.003 | 0.829 |

(6) The effects of Bacteroides fragilis ATCC25285 and vitamin B6 on the expression level of related genes in the jejunum and ileum of laying hens during FIPR are shown in FIGS. 4 and 5. Compared with the control group, in the Bacteroides fragilis ATCC25285+vitamin B6 compound additive group, the expression levels of intestinal barrier-related genes occludin-1 and Claudin-1 and intestinal proliferation-related genes CDK1 and Lgr5 are significantly increased (P<0.05), and the expression levels of intestinal pro-inflammatory-related genes TNF-α, IL-6, and IL-18 are significantly decreased (P<0.05).

The results show that the Bacteroides fragilis ATCC25285+vitamin B6 compound additive is better than the treatment of Bacteroides fragilis ATCC25285 and vitamin B6 alone in the process of FIPR of laying hens. The Bacteroides fragilis ATCC25285+vitamin B6 compound additive can improve the production performance of laying hens during FIPR, improve their intestinal tissue morphology, improve intestinal barrier function and proliferation-related genes, reduce the expression of intestinal inflammation-related genes, and enhance the organism's immune function, thereby maintaining the intestinal health of laying hens.

Therefore, the compound additive and method provided by the present disclosure can significantly increase the ileum villus height of laying hens, the expression levels of intestinal barrier-related genes and intestinal proliferation-related genes, significantly reduce the expression levels of intestinal pro-inflammatory-related genes, and alleviate intestinal stress injury and inflammation, thereby significantly improving the intestinal health of laying hens. And the compound additive and method can significantly increase the expression levels of IgA, IgG and IgM, thereby improving the immune performance of laying hens. With the improvement of intestinal health and immunity, the FIPR of laying hens is successfully achieved: the recovery ability of weight and laying rate of laying hens after fasting, and the color depth of egg yolk on the 30^{th} day of resumption after fasting are significantly better than those of the control group. This provides a technical guarantee for prolonging the breeding period of laying hens, saving costs and increasing efficiency.

Finally, it should be noted that the above embodiments are merely used for describing the technical solutions of the present disclosure, rather than limiting the same. Although the present disclosure has been described in detail with reference to the preferred examples, those of ordinary skill in the art should understand that the technical solutions of the present disclosure may still be modified or equivalently replaced. However, these modifications or substitutions should not make the modified technical solutions deviate from the spirit and scope of the technical solutions of the present disclosure.

This application contains a Sequence Listing XML as a separate part of the disclosure, which presents nucleotide and/or amino acid sequences and associated information using the symbols and format in accordance with the requirements of 37 CFR-1.831-1.835. The XML file named “CNUS—SZ-U-61-2025_seq.xml”, created Jan. 28, 2026, 16,665 bytes in size, is submitted herewith and is incorporated by reference in its entirety.

What is claimed is:

1. A compound additive for fast-induced physiological remodeling (FIPR) of laying hens, wherein the compound additive comprises Bacteroides fragilis ATCC25285 and vitamin B6, and wherein an amount of viable bacteria of Bacteroides fragilis ATCC25285 in the compound additive is 1×10⁹ CFU/mL, and an amount of vitamin B6 in the compound additive is 0.5 mg/mL.

2. The compound additive for FIPR of laying hens according to claim 1, wherein Bacteroides fragilis ATCC25285 is activated with a brain-heart infusion broth medium, and a pH of the medium is 7.4±0.2.

3. A method for FIPR of laying hens, wherein the method makes use of the compound additive for FIPR of laying hens according to claim 1, wherein the method comprises the following steps:
S1, performing a fasting induction by selecting healthy laying hens in a late laying stage, stopping the water and feed supply on days 1-3 of fasting, exposing the laying hens to light for 8 h every day, and avoiding a flow of staff and noise pollution beside a chicken coop containing the healthy laying hens in a late laying stage;
S2, on days 4-15 of fasting, continuously stopping feed supply, supplying water normally, administering the compound additive for FIPR of laying hens, and exposing the laying hens to light for 8 h every day; and
S3, when a weight loss rate of laying hens is equal to or greater than 25%, resuming the feeding for the laying hens, and gradually prolonging a light exposure time for 30 min every day until 16 h, and stopping the prolonging after 16 h.

4. The method for FIPR of laying hens according to claim 3, wherein in S2, 1 mL of the compound additive for FIPR of laying hens is administered to each laying hen.

5. The method for FIPR of laying hens according to claim 3, wherein in S2, the fasting is terminated early when a mortality rate of the laying hens is equal to or greater than 4%.

6. The method for FIPR of laying hens according to claim 3, wherein surroundings of the chicken coop are sterilized daily during fasting.

7. The method for FIPR of laying hens according to claim 3, wherein in S3, a method for resuming the feeding comprises: feeding 30 g of basal diet for each laying hen daily on days 1 and 2 after resuming the feeding; feeding 60 g of basal diet for each laying hen daily on days 3 and 4 after resuming the feeding; feeding 90 g of basal diet for each laying hen daily on day 5 after resuming the feeding; and feeding 120 g of basal diet for each laying hen daily on days 6-20 after resuming the feeding; and after day 20 after resuming the feeding, the laying hens are allowed free feeding.

8. An application for a compound additive for FIPR of laying hens according to claim 1 in the FIPR of laying hens.


SEQUENCE LISTING (18 sequences)

SEQ ID NO: 1 - DNA, 21 bp, synthetic construct
  source 1..21: mol_type = other DNA
gcccagttcagatgagttgcc

SEQ ID NO: 2 - DNA, 20 bp, synthetic construct
  source 1..20: mol_type = other DNA
aagaggccaccacacgacag

SEQ ID NO: 3 - DNA, 23 bp, synthetic construct
  source 1..23: mol_type = other DNA
ctccataggtttccgaggcttgt

SEQ ID NO: 4 - DNA, 22 bp, synthetic construct
  source 1..22: mol_type = other DNA
atgtgttttctgactctccggc

SEQ ID NO: 5 - DNA, 20 bp, synthetic construct
  source 1..20: mol_type = other DNA
aaatgtccagcagccagact

SEQ ID NO: 6 - DNA, 20 bp, synthetic construct
  source 1..20: mol_type = other DNA
ctttcggagcggccttcata

SEQ ID NO: 7 - DNA, 20 bp, synthetic construct
  source 1..20: mol_type = other DNA
tgatgagctggaatgcgatg

SEQ ID NO: 8 - DNA, 21 bp, synthetic construct
  source 1..21: mol_type = other DNA
actgccagatttcacctcctg

SEQ ID NO: 9 - DNA, 20 bp, synthetic construct
  source 1..20: mol_type = other DNA
ccgtggagtccagtgatgag

SEQ ID NO: 10 - DNA, 20 bp, synthetic construct
  source 1..20: mol_type = other DNA
tcgaactcctgcttgtagcg

SEQ ID NO: 11 - DNA, 22 bp, synthetic construct
  source 1..22: mol_type = other DNA
cacccgttaacaccagatttga

SEQ ID NO: 12 - DNA, 20 bp, synthetic construct
  source 1..20: mol_type = other DNA
agggggcatttttggggtag

SEQ ID NO: 13 - DNA, 20 bp, synthetic construct
  source 1..20: mol_type = other DNA
acgtcttgcaggaaatggct

SEQ ID NO: 14 - DNA, 20 bp, synthetic construct
  source 1..20: mol_type = other DNA
ttggcatccaggcgtagaga

SEQ ID NO: 15 - DNA, 19 bp, synthetic construct
  source 1..19: mol_type = other DNA
taatagatgacaaaggggt

SEQ ID NO: 16 - DNA, 19 bp, synthetic construct
  source 1..19: mol_type = other DNA
gagtggaatacagagcaga

SEQ ID NO: 17 - DNA, 20 bp, synthetic construct
  source 1..20: mol_type = other DNA
tgttaccaacacccacaccc

SEQ ID NO: 18 - DNA, 20 bp, synthetic construct
  source 1..20: mol_type = other DNA
tcctgagtcaagcgccaaaa